(19) United States
(12) Patent Application Publication
(10) Pub. No.: US 2006/0105021 A1
(43) Pub. Date: May 18, 2006

(54) Cholesterol-reducing liquid

(75) Inventors: George E. Steele, Fort Worth, TX (US); William E. Steele IV, Fort Worth, TX (US); Donna Henry, Honey Grove, TX (US)

Correspondence Address: DALLAS OFFICE OF FULBRIGHT & JAWORSKI L.L.P., 2200 ROSS AVENUE, SUITE 2800, DALLAS, TX, 75201-2784, US

(73) Assignee: Remington Direct LP, Fort Worth, TX (US)

(21) Appl. No.: 11/233,393

(22) Filed: Sep. 22, 2005

Related U.S. Application Data
(60) Provisional application No. 60/612,733, filed on Sep. 24, 2004.

(51) Int. Cl. A61K 47/00; A61K 31/56; A61K 31/045; A61K 36/68

(52) U.S. Cl. 424/439; 514/171; 514/724; 424/738

(57) ABSTRACT

A dry-blended cholesterol-reducing composition and its method of manufacture are disclosed herein, wherein such cholesterol-reducing composition is formed by adding a dry-blended mixture including cholesterol-lowering active ingredients to a liquid, wherein the dry-blended mixture evenly disperses in the liquid for human consumption. The cholesterol-lowering active ingredients include phytosterols, inositol, policosanol, niacin, omega-3 fatty acids and Fibersol-2®.

### RELATED APPLICATIONS

The present application claims priority to co-pending U.S. Provisional Patent Application No. 60/612,733, entitled “Cholesterol-Reducing Liquid,” filed Sep. 24, 2004, the disclosure of which is hereby incorporated herein by reference.

### TECHNICAL FIELD

The present invention relates generally to the field of cholesterol-reducing compositions, and more particularly, to a composition and method of forming a cholesterol-reducing liquid in the form of either a nutritional supplement or beverage.

### BACKGROUND OF THE INVENTION

Cholesterol has become a concern in that atherosclerosis or coronary heart disease is most often due to atherosclerotic obstruction of large coronary arteries. A risk factor for heart disease is hypercholesterolemia and specifically high blood levels of low density lipoprotein (LDL) cholesterol or low levels of high density lipoprotein (HDL) cholesterol among other factors. When LDL cholesterol levels are decreased, there typically is a decrease in risk of heart disease. Thus, a desire exists to find ways to reduce LDL cholesterol levels in risk subjects, such as through nutritional supplements.

While traditional treatments for hypercholesterolemia are available, there is a preference among some people to use a natural remedy. Examples of natural products shown to be effective in treating hypercholesterolemia are policosanols, phytosterols, inositol, niacin, fiber and omega-3 fatty acids. While each of these cholesterol-reducing natural products are available, there has been little to teach or suggest their combination. One reason for the failure of others to combine these ingredients is that in the past they have only been available in solid form either as a powder or encapsulated as a pill. To get the necessary doses, one would have to take multiple pills on multiple occasions in a single day. This is generally undesirable for a number of reasons including inconvenience and the fact some people encounter problems with solids, such as difficulty in swallowing. While it is generally understood that liquids are more easily ingested and are preferable to many people, combining these known cholesterol-reducing agents has been problematic.

It is known to produce liquid nutritional supplements by batching and processing. This batching and processing is done in multiple stages. For example, a solution may be prepared first, then an acid is added in the next stage, followed by the addition of gums, and so forth. This approach is problematic with respect to cholesterol-reducing compositions in that many of the ingredients forming the liquid cholesterol-reducing composition are hydrophilic in nature, and as the process typically begins with the creation of a solution, ingredients tend to react before it would be preferable for reaction to occur. Accordingly, a need exists for a cholesterol-reducing composition that may be developed to stay in liquid form.

U.S. Pat. No. 6,576,285 discloses a beverage to produce a cholesterol-lowering benefit when consumed with cholesterol-containing meals. However, some ingredients fall out of solution making for an undesirable product. Moreover, no one is known to have successfully combined multiple cholesterol-reducing agents in a single liquid beverage or supplement.

Thus, there exists a need for a cholesterol-lowering beverage or liquid supplement and a method for producing such a cholesterol-lowering beverage or liquid supplement where the active ingredients are evenly dispersed in the liquid and remain in solution after processing.

Another problem that has existed with respect to preparation of a cholesterol-reducing liquid is how to keep some of the fat-soluble ingredients that may be important to production of such a liquid in solution. Ingredients that may be used for lowering cholesterol, such as policosanol and phytosterols, do not combine well in liquid form, as these fat-soluble ingredients tend to repel one another due to their chemical index being different. Thus, there also exists a need for a method of emulsification of these fat-soluble ingredients in order to prepare a cholesterol-lowering beverage or liquid supplement.

### BRIEF SUMMARY OF THE INVENTION

The present invention is directed to a composition and method of producing a cholesterol-reducing beverage and liquid nutritional supplement. According to an embodiment of the invention, a method for producing a cholesterol-reducing liquid includes mixing a first set of ingredients comprising acids, at least one maltodextrin, sweetener, preservatives and colors for a period of time sufficient to disperse the colors, then adding a second set of ingredients comprising propylene glycol alginate, gums, and a first set of active ingredients comprised of cholesterol-reducing agents to the first set of ingredients. This first set of active ingredients includes inositol, niacin and polycosinol. Calcium silicate also may be added in this step as needed. The first and second sets of ingredients are then blended for a predetermined time. A second set of active ingredients comprised of cholesterol-reducing agents then are added to the mixture and blended. This second set of active ingredients comprises Fibersol-2® and phytosterols. Additional maltodextrins may be added with this second set of active ingredients. This dry-blended mixture then is added to a liquid, such as water, and mixed until the dry-blended mixture is dispersed evenly in the liquid.

An embodiment of the cholesterol-reducing composition provides for a liquefied beverage or supplement wherein a dry-blended mixture is added to water and blended until the mixture is evenly dispersed in water. The dry-blended cholesterol-reducing mixture includes active ingredients such as phytosterols, inositol, policosanol, niacin, omega-3 fatty acids and fibers. Other components of the mixture include acids, gums, maltodextrins, as well as other ingredients which may include preservatives, sweeteners, and colors.

Further embodiments of this invention provide a number of technical advantages. Active ingredients useful as aids to lower one's cholesterol are more evenly dispersed in solution using the dry-blend method for preparation of the cholesterol-reducing liquid beverage or supplement. Consequently, a more uniform liquid beverage or supplement may be obtained.

The present invention is further directed to emulsification of fat-soluble ingredients, such as policosanol and phytosterols, in order to produce a cholesterol-lowering beverage or liquid supplement. Agglomerated maltodextrins as well as gums are added to these fat-soluble ingredients in order to place these ingredients into solution.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiments disclosed may be readily utilized as a basis for modifying or designing other structures for carrying out the same purposes of the present invention. It should also be realized that such equivalent constructions do not depart from the invention as set forth in the appended claims. The novel and non-obvious features which are believed to be characteristic of the invention, both as to its organization and method of operation, together with further objects and advantages will be better understood from the following description. It is to be expressly understood, however, that each of the examples is provided for the purpose of illustration and description only and is not intended as a definition of the limits of the present invention.

### DETAILED DESCRIPTION OF THE INVENTION

In one embodiment of the invention, a method of producing a cholesterol-reducing beverage and liquid supplement is provided wherein a dry-blending process is employed. A first set of ingredients are blended together, and this set of ingredients includes acids, at least one maltodextrin, sweetener, preservatives and colors. At least one maltodextrin and acids are preferably combined to provide a base for the dry-ingredient blend when it is later mixed with a liquid, such as water. Maltodextrins and acids are manufacturing aids that help in getting the dry-ingredient blend into suspension when water is introduced and in keeping the ingredients in suspension.

Maltodextrins are mixtures of glucose, maltose, oligosaccharides, and polysaccharides used in the dry-ingredient blend for their digestible carbohydrate qualities, particularly for stabilizing the dry-ingredient blend. Maltodextrins appropriate for addition at this stage of the dry-blending process preferably include maltodextrin 100 ADM CR15. Other maltodextrins are preferably added in later steps, and these maltodextrins include maltodextrin star dri 1015A and maltodextrin star dri 1005D. Maltodextrins preferably comprise 20% of the dry-ingredient blend.

A variety of acids are preferably used to form this composition, including but not limited to citric acid, fumaric acid, and malic acid. These acids typically comprise approximately 1% of the dry-ingredient blend.

These ingredients are blended until the colors are well dispersed, typically over a period of approximately 2-5 minutes, although it should be appreciated that a longer or shorter period of blending may be employed if dispersion occurs slower or more rapidly. Suitable apparatuses for blending include a cone, ribbon blender or a tumbler. Ingredients comprising the first set of ingredients should each be added to the blender separately so as to keep dust migration of the ingredients at a minimum. Although preferably these ingredients are not added in a pre-ground form, these ingredients may be ground prior to blending as desired.

It also should be appreciated that any type of color may be used in this first set of ingredients, as long as the color is suitable for human consumption, and colors may be combined depending on the color desired for the liquid supplement. Examples of preferable colors include, but are not limited to, color yellow #6 alum lake 27%, color yellow #5, and color red #40 alum lake 39%, available from Roha (Haile).

In a preferred embodiment, neotame is preferably introduced as a sweetener. Although neotame is a non-nutritive sweetener, it has been shown to withstand thermal heating better than other sweeteners that tend to affect the coloring of a composition when heating is employed. However, it should be appreciated that other sweeteners may be used without affecting the composition of the dry-ingredient blend that results. Preferable preservatives include sodium benzoate and potassium sorbate.

The following are non-limiting examples of compositions of a preferred first set of ingredients for the dry-blend combination. It should be appreciated that the percentages provided are based on the resulting dry-ingredient blend, and the percentages by weight will change when water is added to the dry blend to produce the liquid supplement.

First Set of Ingredients (Dry-Blend of Cholesterol-Reducing Composition)

0.626% by weight citric acid

0.389% by weight fumaric acid

0.368% by weight malic acid

0.184% by weight colors

7.720% by weight maltodextrin 100 ADM CR15

0.010% by weight neotame

0.261% by weight sodium benzoate

0.261% by weight potassium sorbate

When blending of this first set of ingredients is completed, a second set of ingredients is added to the first set of ingredients, and these two sets of ingredients are blended together. This second set of ingredients includes propylene glycol alginate, gums, and a first set of active ingredients having cholesterol-lowering agents are added. The first set of active ingredients preferably includes inositol, niacin and polycosinol. Each of these active ingredients comprising the first set of active ingredients on its own has been determined to provide cholesterol-lowering effects and will be discussed briefly in turn.

Inositol, as vitamin B3 inositol hexahydroxyeyelohexane, part of the vitamin B-complex, works to prevent accumulation of fats in the liver. Niacin, better known as vitamin B3, also is part of the vitamin B-complex and is a water-soluble vitamin. Niacin has been known to lower blood cholesterol levels.

Policosanol is a mixture of fatty alcohols derived from the wax of honey bees, sugar cane and rice bran and acts to lower cholesterol levels by several mechanisms, including blocking the formation of cholesterol in the liver. Policosanol has been shown to both decrease LDL cholesterol levels and increase HDL cholesterol in the body. Examples of methods for extracting policosanol from sugar cane is described in U.S. Pat. Nos. 5,856,316 and 5,663,156.

A number of gums are preferably incorporated into the dry-ingredient blend. In a preferred embodiment, xanthan gum and organic sodium carboxymethylcellulose (CMC) gum are utilized. These gums are preferably used for binding of ingredients during the blending process.

Propylene glycol alginate is used as a thickener and stabilizer in food products. Calcium silicate also may be added in this step if a flo agent is needed.

The combination of the first and second sets of ingredients are blended, preferably for a period of approximately two minutes. Again, preferable blending devices include a cone, ribbon blender or tumbler. It is important to note that these ingredients should not be over-blended. Further, the blending device used should not continue operation beyond the time needed to blend the first and second sets of ingredients together, as heat generation may occur. This heat generation then may affect the texture of the dry-blend and/or cause incomplete or inaccurate color dispersion.

The following are non-limiting examples of compositions of a preferred second set of ingredients for the dry-blend combination. It should be appreciated that the percentages provided are based on the resulting dry-ingredient blend, and the percentages by weight will change when water is added to the dry blend to produce the liquid supplement.

Second Set of Ingredients (Dry-Blend of Cholesterol-Reducing Composition)

0.652% by weight propylene glycol alginate

1.526% by weight CMC gum

0.600% by weight xanthan gum

First set of active ingredients (0.926% by weight inositol; 0.741% by weight policosanol; 0.376% by weight niacin)

In a further step, additional maltodextrins are preferably added. These maltodextrins include maltodextrin 1015A and maltodextrin 1005A. Also, a second set of active ingredients is added to the combination. The second set of active ingredients preferably is comprised of Fibersol-2® and phytosterols. Each of these active ingredients comprising a second set of active ingredients on its own has been determined to provide cholesterol-lowering effects and will be discussed briefly in turn.

Phytosterols are natural components of edible vegetable oils, such as sunflower seed oil. Phytosterols are effective in lowering LDL cholesterol by inhibiting the absorption of cholesterol from the small intestine. Many foods have been enriched with phytosterols in order to enhance the cholesterol-lowering ability of traditional food products.

Soluble fibers are known for stabilizing blood sugars and reducing serum cholesterol. Fibersol-2® is a soluble dietary fiber available from Matsutani America Inc. and typically comes in the form of a spray-dried powder produced by the pyrolysis and controlled enzymatic hydrolysis of cornstarch. Fibersol-2® improves carbohydrate metabolism, and this results in lowering LDL-cholesterol levels without a change in HDL-cholesterol levels.

The following are non-limiting examples of compositions of a preferred combination of ingredients for this step. It should be appreciated that the percentages provided are based on the resulting dry-ingredient blend.

Composition Including Second Set of Active Ingredients (Dry-Ingredient Blend)

5.190% by weight maltodextrin star dri 1015A

8.607% by weight maltodextrin star dri 1005

16.522% by weight phytosterols

55.044% by weight Fibersol-2®

In this step, each ingredient is added to a blending device. Fibersol-2® preferably should be added first. It should be noted that this set of ingredients is added to a blending device not containing the combination of the first and second sets of ingredients, as this pre-blend is preferably added later. When Fibersol-2® is added, screening should occur in which the blending device should preferably be rotated from side-to-side until all of the Fibersol-2® has been added. Then, phytosterols should be added in a manner similar to how Fibersol-2® was added. The phytosterols and Fibersol-2® then should be blended for approximately 45 seconds, and approximately 20-25 seconds into the blending process, blending should be paused in order to recirculate the dead zones and ensure complete blending of these actives.

Following blending of the phytosterols and Fibersol-2®, maltodextrin 1005A is preferably added to the mixture, and these ingredients should be blended for approximately 30 seconds. Maltodextrin 1015A preferably is added and blending should occur for approximately 30 seconds, pausing the blending process as needed in order to recirculate the dead zones as previously discussed.

Then, the pre-blend of the first and second sets of ingredients should be added to this mixture of maltodextrins, phytosterols and Fibersol-2®. This addition preferably should occur while the blending device is in operation. Rotating addition should be employed until all of the pre-blend has been added. Blending of this resultant mixture preferably occurs until the color has been dispersed through the mixture, and this dispersion results in approximately two minutes.

When dispersion is achieved, the blending device is stopped, and a small amount of the dry-ingredient blend should be removed from the bottom of the blending device and added to the top of the blending device while the blending device is rotated. Further blending is preferably performed for approximately one minute, and an additional recirculation of the blended product should occur as previously discussed.

When blending is completed, a dry-ingredient cholesterol-reducing blend has been formed, and this may be packaged for later mixing with water, for example, to form a liquid supplement as will be described further below.

Accordingly, of the resultant dry-ingredient blend, active ingredients comprise 70 to 80% by weight. Further, the cholesterol-reducing active ingredients comprise 10 to 20% phytosterols, 0.5 to 1.0% inositol, 0.5 to 1.0% policosanol, 0.2 to 0.5% niacin, and 45 to 55% Fibersol-2®.

Approximately 10 to 15% by weight of the dry-ingredient blend is comprised of maltodextrins. The dry-ingredient blend is further comprised of 1 to 2% by weight gums and 1 to 2% by weight acids. Negligible percentages of sweeteners, colors and preservatives form part of the dry-ingredient blend.

When the cholesterol-reducing composition is to be prepared in liquefied form, the dry-ingredient blend is added to water. Under shear agitation, the dry-ingredient blend is slowly added to water, and the blend is mixed with water until all ingredients comprising the blend are dispersed through the water. Shear agitation may be accomplished using a lightening mixer or its equivalent. Depending on what viscosity of the resulting solution is desired, additional mixing may be desired. Accordingly, as all of the dry ingredients have been pre-mixed, the ingredients react with water simultaneously, and this preferably results in even dispersion and dissolution of the ingredients in water.

In further embodiments, the cholesterol-reducing beverage or liquid supplement may include flavorings. These flavorings may include but are not limited to fruit flavorings and chocolate or vanilla flavorings. Flavorings are introduced into water prior to mixing the dry-ingredient blend with water. In this embodiment, water and flavorings are mixed together until the flavorings are dispersed evenly in the water. A cloud also may be added at the same time that the water and flavorings are mixed together.

It also should be appreciated that not all of the water needed to form the liquid blend is preferably present at the time that the dry-blend is added to the water. In an embodiment of the invention, approximately ¾ of the water needed is present in the tank at the time of addition. The remaining volume of water is then preferably added following addition of the dry-blend. When this remaining volume of water is added, then mixing should occur under continual shear agitation for approximately 10-20 minutes or until the dry-blend is evenly dispersed in the water. Preferably, the liquid blend is comprised of at least 75% water. It should be appreciated that if a lower concentration liquid is desired, a higher percentage of water may be added. Similarly, if a higher concentration is desired, it may be preferable for the liquid blend to be comprised of no more than 75% water. However, enough water should be included so that the liquid blend formed is palatable.

When the dry-ingredient blend is added to water and a mixture results, the percentages of active ingredients present in the liquefied supplement are modified, as shown below.

Active Ingredient Composition (After Being Added to Water)

3.177% by weight phytosterols

0.178% by weight inositol

0.142% by weight policosanol

0.072% by weight niacin

10.583% by weight Fibersol-2®

Thus, when the dry-ingredient blend and water are mixed, the cholesterol-reducing active ingredients comprise approximately 10 to 15% by weight of the liquid supplement that is formed. Accordingly, the cholesterol-lowering liquid supplement includes 3 to 4% phytosterols, 0.1 to 0.2% inositol, 0.1 to 0.2% policosanol, 0.05 to 0.1% niacin and 8 to 12% Fibersol-2®.

Maltodextrins comprise approximately 4 to 5% by weight of the liquid supplement. The acids taken as a whole (citric acid, fumaric acid and malic acid combined) comprise 2 to 3% by weight of the liquid supplement, and another 2 to 3% of the liquid supplement are the gums (xanthan and CMC combined). Other ingredients, including flavorings, color, and sweeteners comprise negligible percentages of the liquid supplement that is formed.

Accordingly, the liquid cholesterol-reducing composition that results has a cholesterol-lowering effect. As has been discussed, each of the active ingredients incorporated in the dry-ingredient blend on its own has a cholesterol-lowering effect, and these active ingredients, in combination, provide for an increased chance of reducing the amount of LDL cholesterol found in a human.

The method of dry blending the ingredients prior to introducing the ingredients into solution results in more of the ingredients remaining in solution.

Although the cholesterol-reducing liquid and the method for its production have been described as including a number of formulation aids, it should be appreciated that some of these ingredients may be absent from the dry-blend without changing the cholesterol-reducing effect of the liquid formed. For example, sodium benzoate and potassium sorbate may be omitted from the liquid. Further, sweeteners, flavors, and clouds may be eliminated, although a change in the taste and coloring will result. Additionally, although the invention has been described as including citric acid, fumaric acid and malic acid, other acids may be substituted for these acids without adversely affecting the cholesterol-reducing liquid that results.

It should be further appreciated that although the composition of the dry-blend of ingredients as well as the liquid blend have been described with respect percentages by weight of the resulting composition, the composition of the active ingredients present in the dry-ingredient blend as well as in the cholesterol-reducing liquid should be evaluated in terms of a dose or a recommended daily serving of the liquid. A recommended dose of this liquid is preferably 1 fluid ounce, and a daily serving of this liquid may be as much as 2 fluid ounces; however other quantities constituting a serving may be employed. Accordingly, a daily serving of this liquid, regardless of the serving size, should preferably include 40 mg or greater of policosanol, and preferably within the range of 40-80 mg. A serving may also include 900 mg phytosterols or greater, approximately 20 mg niacin, 50 mg inositol and 3 g of soluble fiber (Fibersol-2®. It should be appreciated that the upper range of the amounts of each of the active ingredients is variable in that the added cholesterol-reducing effect may not be as great as more of an active ingredient is introduced into the liquid.

The present invention is further directed to emulsification of fat-soluble ingredients, such as policosanol and phytosterols, in order to produce a cholesterol-lowering beverage or liquid supplement. As has been shown with respect to the addition of phyterosterols and Fibersol-2®, agglomerated maltodextrins are added when these active ingredients are introduced in order to make it easier to place these typically water-insoluble, fat-soluble ingredients into solution. Further, gums, such as xanthan gum and CMC gum, are introduced into the dry-blend mixture prior to addition of these active ingredients, in part, to allow for emulsification of these fat-soluble active ingredients in order to prepare a cholesterol-lowering beverage or liquid supplement.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method for producing a cholesterol-reducing liquid, said method comprising:
blending a first set of ingredients having at least one acid, maltodextrins, and colors;
adding a second set of ingredients to said first set of ingredients, said second set of ingredients having a sweetener, propylene glycol alginate, at least one gum, and preservatives;
blending said first and second sets of ingredients for a predetermined time;
introducing active ingredients to a mixture of said first and second sets of ingredients to generate a dry-blend of ingredients, wherein said active ingredients are selected from the group consisting of phytosterols, inositol, policosanol, niacin and Fibersol-2®;
adding said dry-blend of ingredients to a liquid; and
mixing said dry-blend of ingredients and said liquid until said dry-blend of ingredients is evenly dispersed in said liquid.

2. The method of claim 1 wherein said active ingredients comprise:
phytosterols and policosanol.

3. The method of claim 2 wherein said active ingredients further comprises omega-3 fatty acids.

4. The method of claim 1 wherein said sweetener is neotame.

5. The method of claim 1 wherein said method further comprises:
introducing calcium silicate to said second set of ingredients.

6. The method of claim 1 wherein said liquid is water.

7. The method of claim 1 wherein said at least one acid is selected from the group consisting of:
citric acid, malic acid and fumaric acid.

8. The method of claim 1 wherein said at least one acid is citric acid, malic acid or fumaric acid.

9. The method of claim 1 wherein said at least one gum is xanthan gum or CMC gum.

10. The method of claim 1 wherein said dry-blend of ingredients is added to said liquid under shear agitation.

11. A cholesterol-reducing composition, said composition comprising:
a dry-blended mixture including cholesterol-lowering active ingredients; and
a liquid, wherein said dry-blended mixture is added to said liquid and said dry-blended mixture evenly disperses in said liquid.

12. The cholesterol-reducing composition of claim 11, said cholesterol-lowering active ingredients comprising:
phytosterols, inositol, policosanol, niacin, and Fibersol-2©.

13. The cholesterol-reducing composition of claim 11 wherein 70 to 80% of said dry-blended mixture is said cholesterol-lowering active ingredients.

14. The cholesterol-reducing composition of claim 11 wherein said cholesterol-lowering active ingredients comprises 10 to 20% phytosterols, 0.5 to 1.0% inositol, 0.5 to 1.0% policosanol, 0.2 to 0.5% niacin, and 45 to 55% Fibersol-2®.

15. The cholesterol-reducing composition of claim 11 wherein said dry-blended mixture further comprises:
acids;
gums; and
maltodextrins.

16. The cholesterol-reducing composition of claim 15 wherein 10 to 15% of said dry-blended mixture is comprised of said maltodextrins.

17. The cholesterol-reducing composition of claim 15 wherein 1 to 2% of said dry-blended mixture is comprised of said gums.

18. The cholesterol-reducing composition of claim 17 wherein said gums are xanthan gum and CMC gum.

19. The cholesterol-reducing composition of claim 15 wherein 1 to 2% of said dry-blended mixture is comprised of said acids.

20. The cholesterol-reducing composition of claim 19 wherein said acids are citric acid, fumaric acid, and malic acid.

21. The cholesterol-reducing composition of claim 11 wherein said dry-blended mixture is packaged for later mixing with said liquid.

22. A cholesterol-reducing liquid comprising:
water sufficient to make said cholesterol-reducing liquid palatable; and
a dry-blended mixture, said mixture comprising 10 to 15% cholesterol-lowering active ingredients, at least 2% maltodextrins, less than 1% gums, and less than 1% acids,
wherein when said dry-blended mixture is added to said water, said dry-blended mixture is evenly dispersed in said water.

23. The cholesterol-reducing liquid of claim 22 said cholesterol-lowering active ingredients comprising:
phytosterols, inositol, policosanol, niacin, and Fibersol-2®.

24. The cholesterol-reducing liquid of claim 22, said cholesterol-lowering active ingredients comprising:
3 to 4% phytosterols, 0.1 to 0.2% inositol, 0.1 to 0.2% policosanol, 0.05 to 0.1% niacin and 8 to 12% Fibersol-2®.

25. The cholesterol-reducing liquid of claim 22, said dry-blended mixture further comprising:
a sweetener;
at least one preservative; and
colors.

26. The cholesterol-reducing liquid of claim 25 wherein said sweetener is neotame.

27. The cholesterol-reducing liquid of claim 25 wherein said at least one preservative comprises:
sodium benzoate and potassium sorbate.

28. The cholesterol-reducing liquid of claim 22, wherein said dry-blended mixture further comprises propylene glycol alginate.

29. The cholesterol-reducing liquid of claim 22 further comprising:
at least one flavoring and a cloud,
wherein said at least one flavoring and said cloud are mixed with said water prior to introducing said dry-blended mixture into said water.

30. The cholesterol-reducing liquid of claim 22 wherein said water comprises at least 75% of said cholesterol-reducing liquid.

31. The cholesterol-reducing liquid of claim 22 wherein said water comprises no more than 75% of said cholesterol-reducing liquid.
